(19) United States
(12) Patent Application Publication
(10) Pub. No.: US 2025/0043330 A1
(43) Pub. Date: Feb. 6, 2025

(54) METHOD FOR MOLECULE QUANTIFICATION

(71) Applicant: CYTESI, INC., Menlo Park, CA (US)

(72) Inventor: Tung-Yu WU, Menlo Park, CA (US)

(21) Appl. No.: 18/793,259

(22) Filed: Aug. 2, 2024

Related U.S. Application Data
(60) Provisional application No. 63/530,450, filed on Aug. 2, 2023.

(51) Int. Cl. C12Q 1/6806; C12Q 1/6851

(52) U.S. Cl. CPC C12Q 1/6806; C12Q 1/6851

(57) ABSTRACT

Provided is a method for molecule quantification. The method comprises: utilizing an electrowetting-on-dielectric (EWOD) device to split a target droplet into a plurality of sub-droplets; performing a quantitative polymerase chain reaction (qPCR) test on at least one of the plurality of sub-droplets within the EWOD device; and obtaining a result of the qPCR test on the at least one of the plurality of sub-droplets to determine whether the result of the qPCR test is positive or negative.

### CROSS-REFERENCE TO RELATED APPLICATIONS

This application claims the benefit and priority to the U.S. Provisional Patent Application No. 63/530,450 filed on 2 Aug. 2023, the entirety of which is incorporated herein by reference.

### TECHNICAL FIELD

The present disclosure generally relates to a method for molecule quantification. More particularly, the present disclosure relates to a quantitative polymerase chain reaction test with an electrowetting-on-dielectric (EWOD) device.

### BACKGROUND

Quantitative polymerase chain reaction (qPCR), also called real-time PCR or quantitative real-time PCR, is a PCR-based technique that couples amplification of a target DNA sequence with quantification of the concentration of that DNA species in the reaction. qPCR is the ability to monitor the progress of the PCR as it occurs, and reactions are characterized by the point in time during cycling when amplification of a target is first detected than the amount of target accumulated after a numbers of cycles. By detection of amplicons during the early exponential phase of PCR, qPCR enables the quantification of the target DNA sequence when these are proportional to the starting template concentration; and coupled with a preceding reverse transcription reaction, qPCR can be used to quantify gene expression. Without prior sequence or known standards of the specific target DNA sequence of interest, qPCR cannot perform absolute quantification of the target DNA, or the result of the quantification may be imprecise.

Digital polymerase chain reaction (dPCR) works by partitioning a sample into many individual real-time PCR reactions; some portion of these reactions contain the target molecule (positive), while others do not (negative). Following PCR analysis, the fraction of negative result is used to generate the absolute quantification of the target molecules in the samples. For droplet digital polymerase chain reaction (ddPCR), this is a digital PCR method utilizing a water-oil emulsion droplet system to divide PCR samples into micro/nano-scaled discrete samples. Both dPCR and ddPCR require a certain number (around 100-10000) of micro-droplets as a sample base and performing PCR on each of the micro-droplets to achieve absolute quantification, which are high cost and time-consuming, and also if the initial amount of molecules is not within a certain range, ddPCR may not work as expected.

Since the pandemic, the need for genetic analysis has become more widespread and intensive. Due to the requirements for quantitative detection, it is necessary to continue to develop a more flexible and precise method for molecule quantification.

### SUMMARY

Some embodiments of the present disclosure provide a method for molecule quantification, comprising: utilizing an electrowetting-on-dielectric (EWOD) device to split a target droplet into a plurality of sub-droplets; performing a quantitative polymerase chain reaction (qPCR) test on at least one of the plurality of sub-droplets within the EWOD device; and obtaining a result of the qPCR test on the at least one of the plurality of sub-droplets to determine whether the result of the qPCR test is positive or negative.

The present disclosure relates to a method for molecule quantification comprising: (a) utilizing an electrowetting-on-dielectric (EWOD) device to split a target droplet into a plurality of sub-droplets; (b) performing a first quantitative polymerase chain reaction (qPCR) test on at least one of the plurality of sub-droplets within the EWOD device; and (c) obtaining a result of the first qPCR test on the at least one of the plurality of sub-droplets to determine whether the result of the first qPCR test is positive or negative.

According to one aspect, wherein performing a first qPCR test comprises calculating a concentration of a target molecule within the at least one of the plurality of sub-droplets, if the concentration surpasses a preset threshold, the result of the first qPCR test on the at least one of the plurality of sub-droplets is positive.

According to one aspect, before utilizing an EWOD device to split a target droplet into a plurality of sub-droplets, the method comprises performing an initial qPCR test on the target droplet within the EWOD device; and obtaining a result of the initial qPCR test on the target droplet to determine whether the result of the initial qPCR test is positive or negative.

According to one aspect, after determining the result of the first qPCR test is positive, the method further comprises the following step: performing a second qPCR test on another of the plurality of sub-droplets.

According to one aspect, after determining the result of the first qPCR test is positive, the method further comprises the following step: performing a digital droplet polymerase chain reaction (ddPCR) test on the tested sub-droplets.

According to one aspect, after determining the result of the first qPCR test is positive, the method further comprises the following step: performing a diagnostics analysis, or biological applications on the tested sub-droplets.

According to one aspect, the biological applications comprise DNA synthesis, protein synthesis or DNA sequencing.

According to one aspect, after determining the result of the first qPCR test is positive, the method further comprises utilizing the EWOD device to split the tested sub-droplets into a plurality of secondary sub-droplets.

According to one aspect, after utilizing the EWOD device to split the tested sub-droplets into a plurality of secondary sub-droplets the method further comprises the following step: performing a third qPCR test on at least one of the plurality of secondary sub-droplets.

According to one aspect, after utilizing the EWOD device to split the tested sub-droplets into a plurality of secondary sub-droplets, the method further comprises the following step: performing a digital droplet polymerase chain reaction (ddPCR) test on the secondary sub-droplets.

According to one aspect, after utilizing the EWOD device to split the tested sub-droplets into a plurality of secondary sub-droplets, the method further comprises the following step: performing a diagnostics analysis or biological applications on the secondary sub-droplets.

According to one aspect, after determining the result of the first qPCR test is positive, the method further comprises emerging at least a portion of the sub-droplets to form at least one target droplets.

According to one aspect, after emerging at least a portion of the sub-droplets to form at least one target droplets, the method further comprises repeating steps (a), (b), and (c) of the method of the present invention.

According to one aspect, after emerging at least a portion of the sub-droplets to form at least one target droplets, wherein the method further comprises the following step: performing a diagnostics analysis or biological applications on the target droplets.

### BRIEF DESCRIPTION OF THE DRAWINGS

The aspects of the present disclosure will become more easily understood from the following detailed description made with reference to the accompanying drawings. It should be noted that, various features may not be drawn to scale. In fact, the sizes of the various features may be increased or reduced arbitrarily for the purpose of clear description.

FIG. 1 is a flowchart showing an example of a process of the quantification of a target sample according to some embodiments.

FIG. 2 is a flowchart showing another example of a process of the quantification of a target sample according to some embodiments.

FIG. 3 is a flowchart showing another example of a process of the quantification of a target sample according to some embodiments.

FIG. 4 is a flowchart showing another example of a process of the quantification of a target sample according to some embodiments.

FIG. 5 is a flowchart showing another example of a process of the quantification of a target sample according to some embodiments.

### DETAILED DESCRIPTION

The following disclosure provides many different embodiments, or examples, for implementing different features of the provided subject matter. Specific examples of components and arrangements are described below to explain certain aspects of the present disclosure. These are, of course, merely examples and are not intended to be limiting. For example, the formation of a first feature over or on a second feature in the description that follows may include embodiments in which the first and second features are formed or disposed in direct contact, and may also include embodiments in which additional features may be formed or disposed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Spatial descriptions, such as “above,” “below,” “up,” “left,” “right,” “down,” “top,” “bottom,” “vertical,” “horizontal,” “side,” “higher,” “lower,” “upper,” “over,” “under” and so forth, are indicated with respect to the orientation shown in the figures, unless otherwise specified. It should be understood that the spatial descriptions used herein are for purposes of illustration only, and that practical implementations of the structures described herein can be spatially arranged in any orientation or manner, provided that the merits of embodiments of this disclosure are not deviated from by such arrangement.

It should be noted that the disclosed technical content must be within the scope covered. At the same time, terms such as “above”, “first”, “second” and “one” quoted in this specification are only for the convenience of description and are not used to limit the scope of implementation of this application. The change or adjustment of the relative relationship shall also be regarded as the implementable scope of the present application without substantive change in the technical content.

In recent years, there has been continuous development and progress in the field of Droplet Microfluidics (DMF). Especially, an electrowetting-on-dielectric (EWOD) based DMF apparatus provides liquid management based on droplets, and allows software-reconfigurable operations on individual droplets, such as movement, combination, splitting and dispensation from reservoirs by manipulating pico-liter to nano-liter scale droplets in electric fields. The DMF application has the ability to precisely manipulate and move small, discrete volumes of fluids. Hence, EWOD is one of the most promising methods to miniaturize analytical tools.

Specifically, the droplet microfluidic apparatus includes electrodes to manipulate or process droplets of fluid (e.g., moving, splitting, merging or heating the droplets) in a defined space. Specifically, each droplet, acting as an independent reactor, allows a wide range of multiple parallel biological and chemical reactions at microscale.

Conventionally, the methods for quantification by qPCR or dPCR testing follow one path to the end and cannot dynamically adjust subsequent experiments based on experiment results, resulting in a waste of time and the need for additional biological samples and reagents to complete testing.

According to one aspect of the present application, methods or operations described herein such as qPCR, dPCR, genetic analysis, DNA synthesis, protein synthesis or DNA sequencing may all be implemented in the EWOD device. In some embodiments, the process of each operation may be arranged and modified by software in the EWOD device without setting up a new equipment. In some embodiments, preliminary testing and subsequent testing/operations may be performed on a single platform during the whole quantification process, reducing human interference and automating the process from beginning to end.

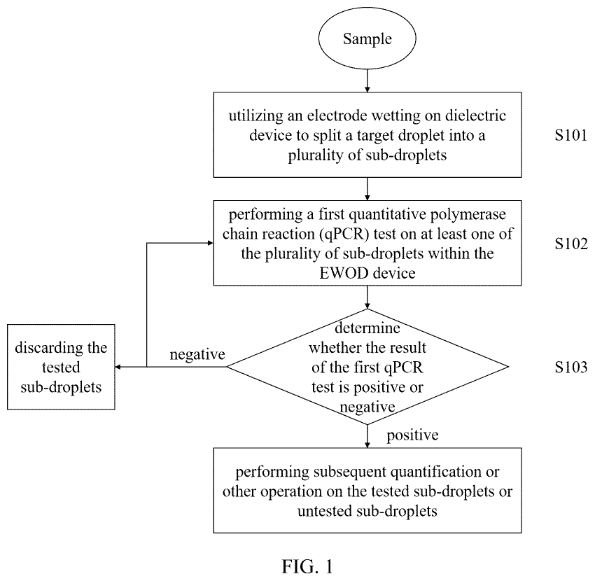

FIG. 1 is a flowchart showing an example of a process of the quantification of a target sample according to some embodiments. In this example, the method comprises the following steps:

S101: utilizing an electrowetting-on-dielectric (EWOD) device to split a target droplet into a plurality of sub-droplets.

As aforementioned, the EWOD device may be implemented to split a droplet into a plurality of micron-sized sub-droplets, including the target droplet used in some embodiments of the present application. In some embodiments, the EWOD device may include an electrode layer, a dielectric layer, and a hydrophobic layer arranged in sequence from bottom to top. The target droplets are set on top of the hydrophobic layer. Voltage may be applied to the electrode layer and the droplets respectively to adjust the surface tension of the droplets over the hydrophobic layer, and then the droplets may be moved or split by applying voltage at different positions on the electrode layer.

In some embodiments, the EWOD device may be implemented to split a droplet containing a target molecule to be detected into sub-droplets of a preset number and volume. The number and volume of the target droplet and sub-droplet can be adjusted according to the requirements of the quantitative analysis. In some embodiments, the volume scale of a split target droplet is micron-liter to nano-liter. In some embodiments, the volume of the sub-droplets is down to the nano-liter range. In some embodiments, the number of sub-droplets may be determined by the applications or operations to which the sub-droplets are subjected according to the requirements of the quantitative analysis. For example, for qPCR testing, the number of the droplet may be preferably in the range of 1-100; and, for ddPCR testing, the number of the droplet may be preferably in the range of 1,000-1,000,000. In some embodiments, the number of sub-droplets is not particularly limited, and may be in the range of 2-1,000,000, 5-100,000, 10-10,000, 100-1,000, or 100-100,000.

In some embodiments, the target droplets may be biological samples. The target droplets may be blood. The target droplets may include DNA, RNA, genes or the like. The target droplets may include one or more of the following materials: PBS, cell culture medium, enzyme, antibody, saliva, blood plasma, DMSO, serum, protein solution, ion solution, magnetic beads solution, SDS, PEG wash buffer, lyse buffer, elution buffer, FBS in DMEM, proteinase K, MgCl2 solution, TE buffer, PCR buffer, PCR primer and/or ethanol.

S102: performing a qPCR test on at least one of the plurality of sub-droplets within the EWOD device.

In some embodiments, a qPCR test may be designed and performed according to the target molecule for quantification, which may be any suitable or known qPCR test procedure in the art. In some embodiments, reagents or compounds of a qPCR primer or probes may be added or mixed directly into the sub-droplets through the EWOD device. In some embodiments, the process for the qPCR test can be controlled and configured by the EWOD device through software to achieve a simple and flexible qPCR test on single or multiple sub-droplets.

S103: obtaining a result of the qPCR test on the at least one of the plurality of sub-droplets to determine whether the result of the qPCR test is positive or negative.

In some embodiments, the result of qPCR test is determined according to the experiment requirements of the target molecule for quantification. In some embodiments, by performing a qPCR test on a portion of sub-droplets, it is possible to perform a preliminary analysis to determine whether the target molecules that need to be quantified and analyzed are present or reach a certain criteria in the target droplet and the tested sub-droplet, and dynamically adjust the quantification process according to the result of the preliminary analysis. In some embodiments, the qPCR test can be performed by fluorescence detection or any known detection method in the art to determine whether the target molecule is present. In some embodiments, if the concentration of a target molecule within the at least one of the plurality of sub-droplets after the qPCR test surpasses a preset threshold after the qPCR test, the result of the qPCR test on at least one of the plurality of sub-droplets may be defined as positive; alternatively, the result of the qPCR test may be defined as negative.

In some embodiments, if the result of the qPCR test on the tested sub-droplet is positive, the remaining sub-droplets or the tested sub-droplets may continue to be subjected to a subsequent more accurate measurement for quantification, such as dPCR or ddPCR, or other biological operations, such as, diagnostics analysis, DNA sequencing, DNA synthesis or protein synthesis. In some embodiments, if the result of the qPCR test on the tested sub-droplet is positive, the tested sub-droplets may be marked as complete and the result may be collected for quantitative analysis.

In some embodiments, if the result of the qPCR test on a portion of the tested sub-droplets is positive and on a portion of the test sub-droplets is negative, it may be used to determine according to the experiment requirements of the target molecule for quantification whether to continue to perform dPCR for absolute quantitative analysis of the tested droplet which result is positive, or to perform qPCR for further testing or other experiments. In some embodiments, if the result of the qPCR test on a portion of the tested sub-droplets is negative, step S102 may be repeated to select other untested sub-droplets for a second qPCR test to determine whether the other untested sub-droplets meet the quantification requirements.

In some embodiments, if the result of the qPCR test on the tested sub-droplet is negative, the tested sub-droplet may be discarded or collected for further analysis based on test requirement.

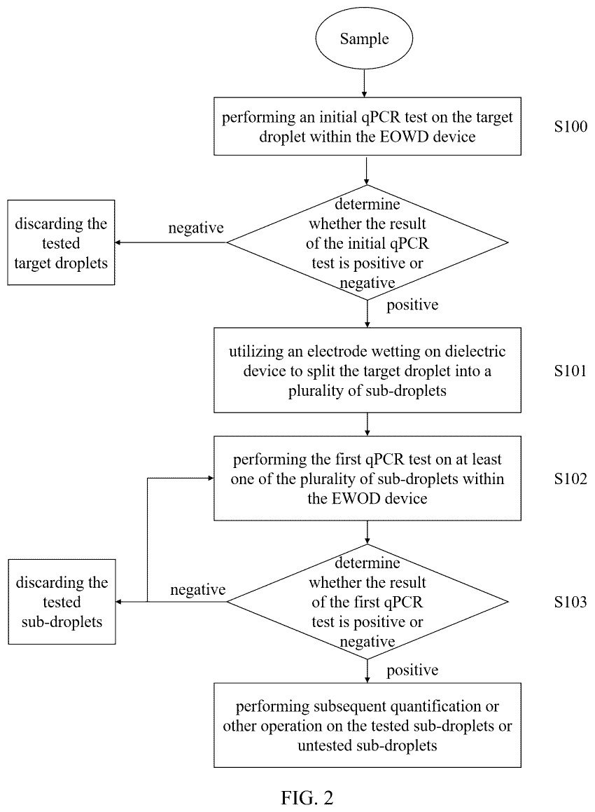

FIG. 2 is a flowchart showing another example of a process of the quantification of a target sample according to some embodiments. In this example, before utilizing the EWOD device to split a target droplet into a plurality of sub-droplets, the method further comprises the step:

S100: performing a first qPCR test on the target droplet within the EWOD device; and obtaining a result of the first qPCR test on the target droplet to determine whether the result of the qPCR test is positive or negative.

In some embodiments, the result of the first qPCR test is determined according to the experiment requirements of the target molecule for quantification. In some embodiments, the first qPCR test prior to the split process may provide a preliminary analysis to determine whether the target molecules that need to be quantified and analyzed are present or reach a certain criteria in the target droplet, thereby determining whether to perform subsequent splitting or quantitative testing on the target droplet. In some embodiments, if the result of the first qPCR test is positive, the target droplet may continue to be subjected to subsequent steps S101˜S103; if the result of the first qPCR test is negative, the target droplet may be discarded.

In some embodiments, if the result of the first qPCR shows that the content of the target molecule in the target droplet is much lower than an expected value, such as 10%, 1% or less, the quantification will be terminated early to reduce cost and time.

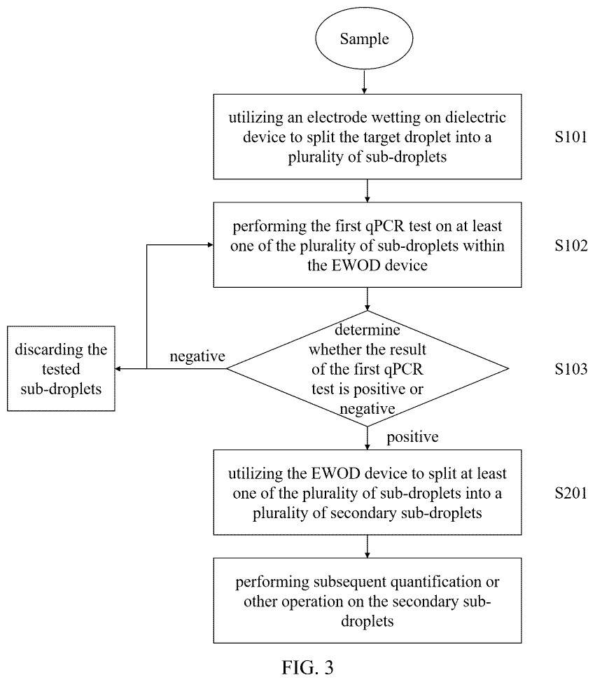

FIG. 3 is a flowchart showing another example of a process of the quantification of a target sample according to some embodiments. In this example, after the result of the qPCR test on the tested droplet being determined as positive, the method further comprises the following steps:

S201: utilizing the EWOD device to split at least one of the tested sub-droplets into a plurality of secondary sub-droplets.

In some embodiments, the tested sub-droplets may be split through the EWOD device into a plurality of secondary sub-droplets for various application so as to increase the number of positive samples for quantitative analysis. In some embodiments, a portion of the secondary sub-droplets may be diluted by or mixed with another liquid(s)/reagents(s)/compound(s) to form new sample droplets for a new set of quantitative analysis.

In some embodiments, a portion of the secondary sub-droplets may continue to be subjected to a subsequent more accurate measurement for quantification, such as dPCR or ddPCR, or other analytical operations, such as, diagnostics analysis, DNA sequencing, DNA synthesis or protein synthesis. In some embodiments, a third qPCR test may be performed on a portion of the secondary sub-droplets to determine the status of the portion of the secondary sub-droplets according to the experiment requirement. In some embodiments, if the result of the third qPCR test on a portion of the secondary sub-droplets is positive, the tested secondary sub-droplets or untested secondary sub-droplets may be further split into a plurality of tertiary sub-droplets according to experiment requirement. In some embodiments, the splitting and testing of a target droplet may be dynamically determined, arranged or terminated based on the result of each qPCR test on the droplets at a different stage during the quantification.

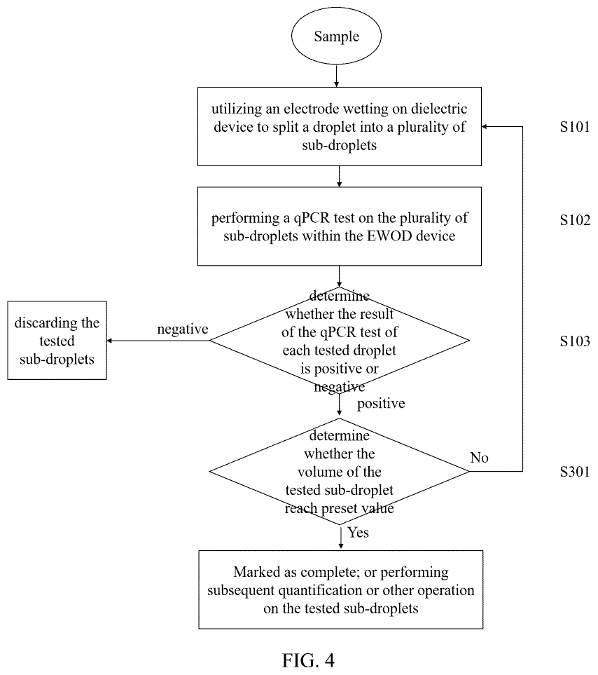

FIG. 4 is a flowchart showing another example of a process of the quantification of a target sample according to some embodiments. In this example, after the result of the qPCR test on the tested droplet being determined as positive, the method further comprises the following steps:

S301: determine whether the volume of the tested sub-droplets reaches a preset value, and if not, repeat steps S101-S103 until the volume of the tested sub-droplets reaches a preset value.

In some embodiments, quantitative analysis requires the target droplet to be within a specific range of volume. Conventionally, if the specific volume of the target droplets is down to nano-liter, directly splitting the sample into a plurality of target droplets may generate a large number of target droplets, in which a portion of the target droplets may generate a negative or error result for quantification of the target molecule depending on the distribution and unity of target molecules in each of the target droplets during the splitting process. In some embodiments, the sample droplet may be split and then qPCR tested to determine whether the separated sub-droplets contain the amount of target molecules as expected, and the sub-droplets with a positive result may be continuously split and qPCR tested until a test droplet with a volume within a specified range is obtained, wherein the number of droplets split can be controlled by the EWOD device to limit the number of qPCR tests each time, and the status of the test droplets can also be controlled, thereby greatly optimizing the quantitative analysis of the target molecule within the test droplets.

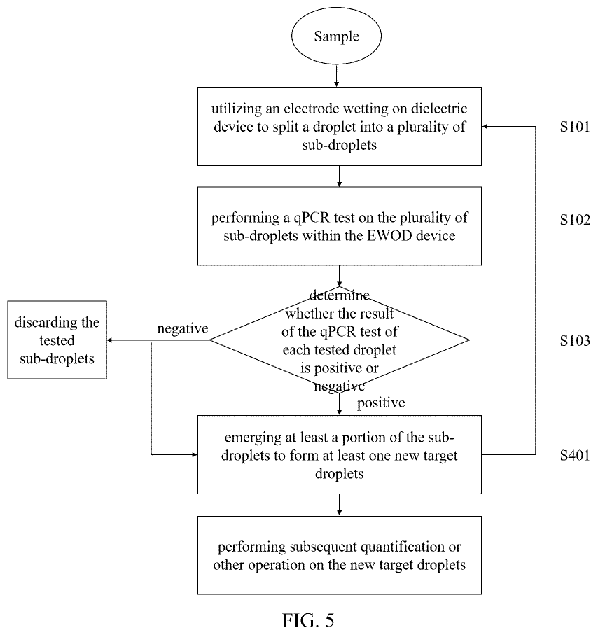

FIG. 5 is a flowchart showing another example of a process of the quantification of a target sample according to some embodiments. In this example, the method comprises the following steps:

S401: emerge the sub-droplets to form at least one target droplet.

In some embodiments, a portion of the sub-droplets may be emerged through the EWOD device to form one or more target droplets with larger volume for various biological operations according to the requirements of the quantitative analysis. For example, some biological operations may require sample droplet with certain volume, by emerging the sub-droplets, including tested sub-droplets and/or untested sub-droplets, the method provides the capability to generate target droplets with higher volume for various operations, including, but not limited to, spiting dPCR or ddPCR, or other analytical operations, such as, diagnostics analysis, DNA sequencing, DNA synthesis or protein synthesis.

In some embodiment, all of the sub-droplets may be emerged into a new target droplet, and the method may repeatedly execute the process of splitting, qPCR testing, and emerging of the target droplet to perform multiple quantitative analysis of a sample droplet for the quantification of the target molecule. In some embodiments, the tested droplet with positive result may be emerged to form one or more target droplets. In some embodiments, the un-tested droplet may be emerged to form one or more target droplets. In some embodiments, the tested droplet with negative result may be emerged to form one or more target droplets. In some embodiments, the sub-droplets to be emerged may be selectable according to the requirements of the quantitative analysis.

In some embodiments, the emerged droplets may be diluted by or mixed with another liquid(s)/reagents(s)/compound(s) to form new sample droplets for a new set of quantitative analysis.

In some embodiments, the quantification of the target molecule in the present application may be dynamically adjusted according to results after each splitting and qPCR testing to provide flexibility and accuracy for quantification.

The foregoing outlines features of several embodiments and detailed aspects of the present disclosure. The embodiments described in the present disclosure may be readily used as a basis for designing or modifying other processes and structures for carrying out the same or similar purposes and/or achieving the same or similar advantages of the embodiments introduced herein. Such equivalent constructions do not depart from the spirit and scope of the present disclosure, and various changes, substitutions, and alterations may be made without departing from the spirit and scope of the present disclosure.

1. A method for molecule quantification, comprising:
(a) utilizing an electrowetting-on-dielectric (EWOD) device to split a target droplet into a plurality of sub-droplets;
(b) performing a first quantitative polymerase chain reaction (qPCR) test on at least one of the plurality of sub-droplets within the EWOD device; and
(c) obtaining a result of the first qPCR test on the at least one of the plurality of sub-droplets to determine whether the result of the first qPCR test is positive or negative.

2. The method of claim 1, wherein performing a first qPCR test comprises calculating a concentration of a target molecule within the at least one of the plurality of sub-droplets, if the concentration surpasses a preset threshold, the result of the first qPCR test on the at least one of the plurality of sub-droplets is positive.

3. The method of claim 1, wherein, before utilizing an EWOD device to split a target droplet into a plurality of sub-droplets, the method comprises:
performing an initial qPCR test on the target droplet within the EWOD device; and
obtaining a result of the initial qPCR test on the target droplet to determine whether the result of the initial qPCR test is positive or negative.

4. The method of claim 1, wherein, after determining the result of the first qPCR test is positive, the method further comprises the following step:
performing a second qPCR test on another of the plurality of sub-droplets.

5. The method of claim 1, wherein, after determining the result of the first qPCR test is positive, the method further comprises the following step:
performing a digital droplet polymerase chain reaction (ddPCR) test on the tested sub-droplets.

6. The method of claim 1, wherein, after determining the result of the first qPCR test is positive, the method further comprises the following step:
performing a diagnostics analysis, or biological applications on the tested sub-droplets.

7. The method of claim 6, wherein the biological applications comprise DNA synthesis, protein synthesis or DNA sequencing.

8. The method of claim 1, wherein, after determining the result of the first qPCR test is positive, the method further comprises:
utilizing the EWOD device to split the tested sub-droplets into a plurality of secondary sub-droplets.

9. The method of claim 8, wherein the method further comprises the following step:
performing a third qPCR test on at least one of the plurality of secondary sub-droplets.

10. The method of claim 8, wherein the method further comprises the following step:
performing a digital droplet polymerase chain reaction (ddPCR) test on the secondary sub-droplets.

11. The method of claim 8, wherein the method further comprises the following step:
performing a diagnostics analysis or biological applications on the secondary sub-droplets.

12. The method of claim 11, wherein the biological application comprises DNA synthesis, protein synthesis or DNA sequencing.

13. The method of claim 1, wherein, after determining the result of the first qPCR test is positive, the method further comprises:
emerging at least a portion of the sub-droplets to form at least one target droplets.

14. The method of claim 13, wherein the method further comprises repeating steps (a), (b), and (c) as defined in claim 1.

15. The method of claim 13, wherein the method further comprises the following step:
performing a diagnostics analysis or biological applications on the target droplets.

16. The method of claim 15, wherein the biological application comprises DNA synthesis, protein synthesis or DNA sequencing.

17. The method of claim 1, wherein the volume of the sub-droplets is down to the nano-liter range.
